(19) United States
(12) Patent Application Publication
(10) Pub. No.: US 2020/0085358 A1
(43) Pub. Date: Mar. 19, 2020

(54) BIOLOGICAL-INFORMATION MEASURING APPARATUS AND NON-TRANSITORY COMPUTER READABLE MEDIUM

(71) Applicant: FUJI XEROX CO., LTD., Tokyo (JP)

(72) Inventors: Kazuhiro SAKAI, Kanagawa (JP); Kazuyuki MATSUSHITA, Kanagawa (JP); Hideaki OZAWA, Kanagawa (JP); Hideyuki UMEKAWA, Kanagawa (JP); Kouhei YUKAWA, Kanagawa (JP); Manabu AKAMATSU, Kanagawa (JP)

(73) Assignee: FUJI XEROX CO., LTD., Tokyo (JP)

(21) Appl. No.: 16/543,622

(22) Filed: Aug. 19, 2019

(30) Foreign Application Priority Data
Sep. 19, 2018 (JP) 2018-175033

(51) Int. Cl. A61B 5/1455; A61B 5/026

(52) U.S. Cl. CPC A61B 5/14551; A61B 5/0059; A61B 5/0261

(57) ABSTRACT

A biological-information measuring apparatus includes an informing unit that informs a subject whose biological information is to be measured of a reference rhythm that is predetermined as a rhythm suitable for measurement of the biological information, a measured value of which varies in accordance with a breathing state of the subject, in such a manner that a breathing rhythm of the subject becomes close to the reference rhythm and a measuring unit that measures the biological information of the subject who performs breathing to the reference rhythm, which is given to the subject by the informing unit.

### CROSS-REFERENCE TO RELATED APPLICATIONS

This application is based on and claims priority under 35 USC 119 from Japanese Patent Application No. 2018-175033 filed Sep. 19, 2018.

### BACKGROUND

### (i) Technical Field

The present disclosure relates to a biological-information measuring apparatus and a non-transitory computer readable medium.

### (ii) Related Art

Japanese Unexamined Patent Application Publication No. 2006-231012 discloses a method of measuring a circulation time of oxygen transportation, the method being used in an apparatus that calculates, by using sensors, changes in oxygen saturation on the basis of a light absorption signal of arterial blood extracted from a living body. In the method, the amount of oxygen to be inhaled by a living body is changed, and the time elapsed from a reference point that is a point in time when the amount of oxygen to be inhaled by the living body is changed to a point in time when the oxygen saturation of arterial blood changes is measured.

Japanese Unexamined Patent Application Publication No. 2010-5192 discloses a medical imaging apparatus that is provided with an instruction display device, which displays an instruction to a patient to breathe or not to breathe, the medical imaging apparatus including a measuring device that measures the elapsed time from the beginning of imaging, a determining device that determines, on the basis of the measurement result obtained by the measuring device, whether the remaining breath-holding time has reached a predetermined period of time, and a control device that starts controlling the instruction display device on the basis of the determination result obtained by the determining device.

In recent years, development of a measurement method of measuring biological information from a value such as, for example, an oxygen circulation time, that indicates the concentration of oxygen in blood has been advanced.

A value that indicates the concentration of oxygen in blood varies in accordance with the breathing state of a subject, and thus, in order to accurately measure biological information, a subject may be led to start inhaling and exhaling in such a manner that the breathing state of the subject becomes close to a rhythm suitable for measurement of the biological information.

### SUMMARY

Aspects of non-limiting embodiments of the present disclosure relate to providing a biological-information measuring apparatus and a biological-information measuring program capable of improving the accuracy with which biological information, the measured value of which varies in accordance with the breathing state of a subject, is measured compared with the case where a subject is allowed to freely breathe of their own will.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided a biological-information measuring apparatus including an informing unit that informs a subject whose biological information is to be measured of a reference rhythm that is predetermined as a rhythm suitable for measurement of the biological information, a measured value of which varies in accordance with a breathing state of the subject, in such a manner that a breathing rhythm of the subject becomes close to the reference rhythm and a measuring unit that measures the biological information of the subject who performs breathing to the reference rhythm, which is given to the subject by the informing unit.

### BRIEF DESCRIPTION OF THE DRAWINGS

Exemplary embodiments of the present disclosure will be described in detail based on the following figures, wherein:

FIG. 1 is a schematic diagram illustrating an example of measurement of oxygen saturation in blood;

FIG. 2 is a graph illustrating an example of changes in the amount of light absorbed by a living body;

FIG. 3 is a graph illustrating examples of the amount of light absorbed by oxyhemoglobin and the amount of light absorbed by reduced hemoglobin at different wavelengths;

FIG. 4 is a diagram illustrating a configuration example of a biological-information measuring apparatus according to a first exemplary embodiment;

FIG. 5 is a diagram illustrating an arrangement example of light-emitting elements and a light-receiving element;

FIG. 6 is a diagram illustrating another arrangement example of the light-emitting elements and the light-receiving element;

FIG. 7 is a graph illustrating an example of changes in the oxygen saturation in blood associated with stoppage of breathing and resumption of breathing;

FIG. 8 is a diagram illustrating a configuration example of a principal portion in an electrical system of the biological-information measuring apparatus according to the first exemplary embodiment;

FIG. 9 is a flowchart illustrating an example of the flow of measurement processing according to the first exemplary embodiment;

FIG. 10 includes a graph illustrating the breathing state of a subject and a graph illustrating an example of changes in oxygen saturation;

FIG. 11 is a diagram illustrating examples of the way of indicating a reference breathing rhythm;

FIG. 12 is a diagram illustrating a configuration example of a principal portion in an electrical system of a biological-information measuring apparatus according to a second exemplary embodiment; and

FIG. 13 is a flowchart illustrating an example of the flow of measurement processing according to the second exemplary embodiment.

### DETAILED DESCRIPTION

Exemplary embodiments of the present disclosure will be described below with reference to the drawings. Note that, in the drawings, components having the same function are denoted by the same reference sign, and steps having the same function are denoted by the same reference sign. Repeated descriptions will be avoided.

### First Exemplary Embodiment

A biological-information measuring apparatus 10 is an apparatus that measures information (biological information) related to a living body 8, particularly biological information related to a circulatory system. A circulatory system collectively refers to a group of organs that transport a body fluid such as, for example, blood while circulating the body fluid within a body.

There are a plurality of types of biological information related to the circulatory system, and an example of a value that indicates the state of the heart that pumps blood into blood vessels is a cardiac output (CO) that represents the volume of blood pumped by the heart.

It is known that, when a cardiac output falls below a reference value, for example, left-sided heart failure is suspected, and when a cardiac output increases above the reference value, for example, right-sided heart failure is suspected. In this manner, a cardiac output is used in various examinations for heart disease and determination of medication effects.

An example of a method of measuring cardiac output is as follows. A catheter with a balloon attached to the tip thereof is inserted into the pulmonary artery of a subject whose cardiac output is to be measured, and the oxygen saturation in blood is measured while the balloon is inflated and deflated. Then, a cardiac output is calculated from the measured oxygen saturation. Here, the oxygen saturation in blood is an example of a value that indicates the concentration of oxygen in the blood and is a value that indicates how much hemoglobin in the blood is bound to oxygen. This value also indicates that a symptom such as, for example, anemia is more likely to occur as the oxygen saturation in the blood decreases.

However, in such a method of measuring cardiac output by using a catheter, since a catheter needs to be inserted into a blood vessel of a subject, it is necessary to perform a surgical procedure, and this method is more invasive to the subject than other measurement methods.

Accordingly, in order to reduce the burden on a subject to be less than the burden that will be imposed on the subject in the case of employing the method of measuring cardiac output by using a catheter, research has been conducted on a method of measuring cardiac output by using oxygen saturation that is determined from the pulse wave of a subject. Pulse wave is a value that indicates changes in pulsation of a blood vessel in response to pumping of blood by the heart.

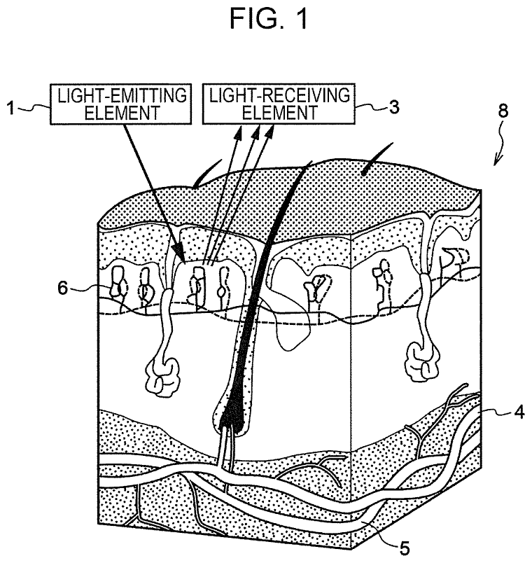

A method of measuring oxygen saturation in blood, which is one of biological information, will be described first with reference to FIG. 1.

As illustrated in FIG. 1, light is radiated from a light-emitting element 1 toward a subject's body (living body 8), and the intensity of light that is reflected by an artery 4, a vein 5, a capillary 6, and the like, which are distributed throughout the body of the subject, and that is received by a light-receiving element 3 or the intensity of light that passes through the artery 4, the vein 5, the capillary 6, and the like and that is received by the light-receiving element 3 (i.e., the amount of reflected light received by the light-receiving element 3 or the amount of transmitted light received by the light-receiving element 3) is used for measuring the oxygen saturation in blood.

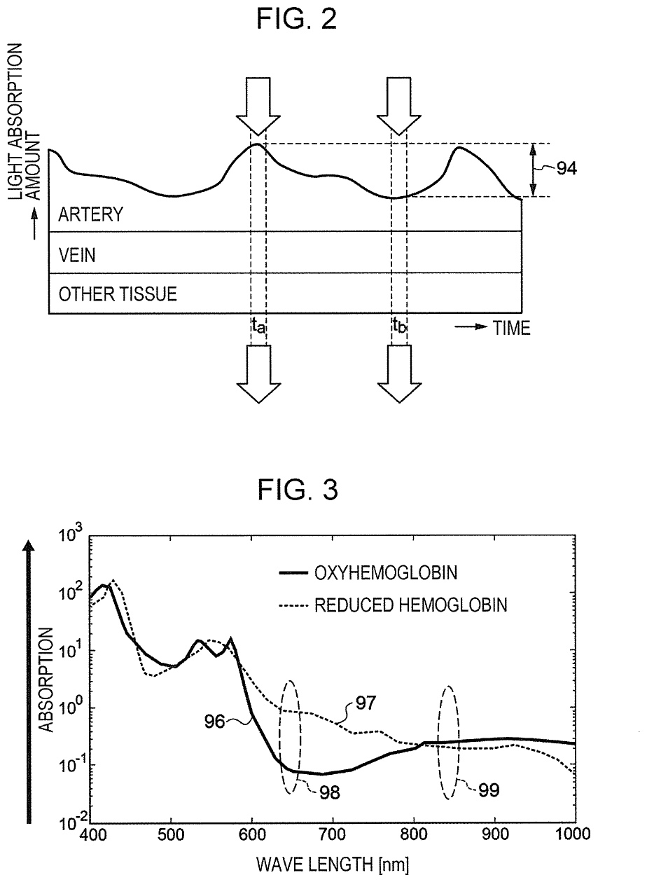

FIG. 2 is a conceptual diagram illustrating, for example, the amount of change in the amount of light that is absorbed by the living body 8. As illustrated in FIG. 2, there is a tendency for the light absorption amount of the living body 8 to fluctuate over time.

In addition, looking at the breakdown of the fluctuations in the light absorption amount of the living body 8, it is known that the light absorption amount of the artery 4 more fluctuates than the light absorption amount of the vein 5 and the light absorption amount of other tissue including stationary tissue, and the degree of fluctuations in each of the light absorption amounts of the vein 5 and the other tissue is vanishingly small as if these light absorption amounts do not fluctuate. This is because the arterial blood that is pumped by the heart flows inside a blood vessel with a pulse wave, and thus, the artery 4 expands and contracts with time in the cross-sectional direction of the artery 4, which in turn results in changes in the thickness of the artery 4. Note that the range indicated by arrow 94 in FIG. 2 represents the degree of fluctuations in the light absorption amount that corresponds to the changes in the thickness of the artery 4.

In FIG. 2, when a light receiving amount at time tₐ is denoted by Iₐ, and a light receiving amount at time t_{b} is denoted by I_{b}, a change ΔA of light absorption amount due to changes in the thickness of the artery 4 may be expressed by Formula (1).

(Math. 1)

ΔA=ln(I_{b}/Iₐ) (1)

In contrast, FIG. 3 is a graph illustrating examples of the amount of light absorbed by hemoglobin (oxyhemoglobin) that has been bound to oxygen flowing inside the artery 4 and the amount of light absorbed by hemoglobin (reduced hemoglobin) that is not bound to oxygen at different wavelengths. In FIG. 3, a line 96 represents the amount of light absorbed by oxyhemoglobin, and a line 97 represents the amount of light absorbed by reduced hemoglobin.

As illustrated in FIG. 3, it is known that oxyhemoglobin is more likely than reduced hemoglobin to absorb light in an infrared (IR) region 99 having a wavelength around about 850 nm, and reduced hemoglobin is more likely than oxyhemoglobin to absorb light, particularly light in a red region 98 having a wavelength around about 660 nm.

It is also known that oxygen saturation and the rate of the change ΔA of light absorption amount at different wavelengths have a proportional relationship.

Accordingly, infrared light (IR light) and red light with which the difference in light absorption amount between oxyhemoglobin and reduced hemoglobin is more likely to become noticeable than with a combination of other wavelengths are used to calculate the ratio between a change ΔA_{IR} of light absorption amount in the case where the IR light is radiated onto the living body 8 and a change ΔA_{Red} of light absorption amount in the case where the red light is radiated onto the living body 8, so that an oxygen saturation S is calculated by using Formula (2). Note that, in Formula (2), k stands for the constant of proportionality.

(Math. 2)

S=K(ΔA_{Red}/ΔA_{IR}) (2)

In other words, when the oxygen saturation in blood is calculated, a plurality of light-emitting elements 1 radiate light beams having different wavelengths onto the living body 8. More specifically, the living body 8 is irradiated by the light-emitting element 1 that emits IR light and the light-emitting element 1 that emits red light. In this case, although a light emission period of the light-emitting element 1 that emits the IR light and a light emission period of the light-emitting element 1 that emits the red light may overlap each other, it is desirable that these light-emitting elements 1 emit the different light beams in such a manner that their light emission periods do not overlap each other. Then, reflected light that is formed of the light beams that have been emitted by the light-emitting elements 1 and reflected by the living body 8 or transmitted light that is formed of the light beams that have been emitted by the light-emitting elements 1 and that have passed through the living body 8 is received by the light-receiving element 3, and Formula (1), Formula (2), or commonly known formulas that are obtained by modifying Formula (1) and Formula (2) are calculated by using the light receiving amount at each light-receiving point in time, so that the oxygen saturation is measured.

As a commonly known formula that is obtained by modifying Formula (1), for example, the change ΔA of light absorption amount may be expressed as in Formula (3) by expanding Formula (1).

(Math. 3)

ΔA=ln I_{b}−ln Iₐ (3)

In addition, Formula (1) may be modified as Formula (4).

(Math. 4)

ΔA=ln(I_{b}/Iₐ)=ln(1+(I_{b}−Iₐ)/Iₐ) (4)

In general, since the formula “(I_{b}−Iₐ)<<Iₐ” holds true, the formula “ln(I_{b}/Iₐ)≈(I_{b}−Iₐ)/Iₐ” also holds true, and thus, Formula (5) may be used instead of Formula (1) as the change ΔA of light absorption amount.

(Math. 5)

ΔA≈(I_{b}−Iₐ)/Iₐ (5)

In the following description, when it is necessary to distinguish the light-emitting element 1 that emits the IR light and the light-emitting element 1 that emits the red light from each other, the light-emitting element 1 that emits the IR light will be referred to as “light-emitting element 1A”, and the light-emitting element 1 that emits the red light will be referred to as “light-emitting element 1B”.

In the above-described method, the oxygen saturation in blood is measured by bringing the light-emitting elements 1 and the light-receiving element 3 close to a body surface of a subject, and thus, the burden on the subject is less than the burden that will be imposed on the subject in the case where the oxygen saturation in blood is measured by inserting a catheter into a blood vessel.

The biological-information measuring apparatus 10 calculates a cardiac output by a method that will be described later using measured oxygen saturation of a subject.

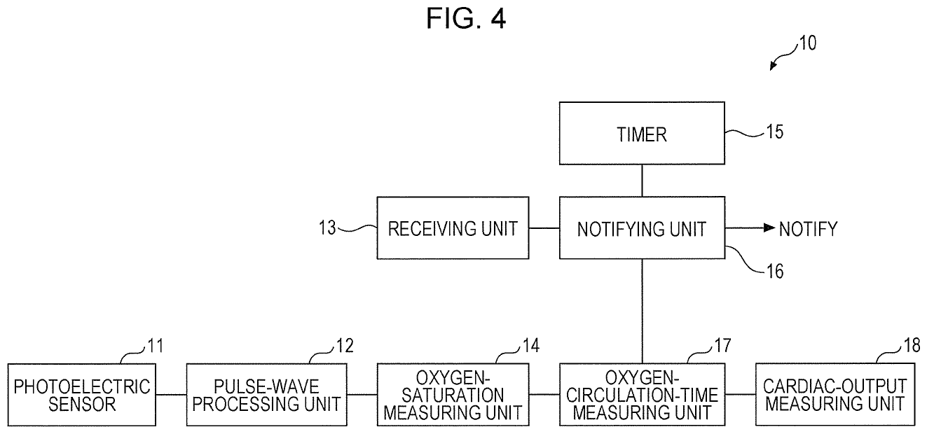

FIG. 4 is a diagram illustrating a configuration example of the biological-information measuring apparatus 10. As illustrated in FIG. 4, the biological-information measuring apparatus 10 includes a photoelectric sensor 11, a pulse-wave processing unit 12, a receiving unit 13, an oxygen-saturation measuring unit 14, a timer 15, and a notifying unit 16, an oxygen-circulation-time measuring unit 17, and a cardiac-output measuring unit 18.

The photoelectric sensor 11 includes the light-emitting element 1A that emits IR light having a central wavelength of about 850 nm, the light-emitting element 1B that emits red light having a central wavelength of about 660 nm, and the light-receiving element 3 that receives the IR light and the red light.

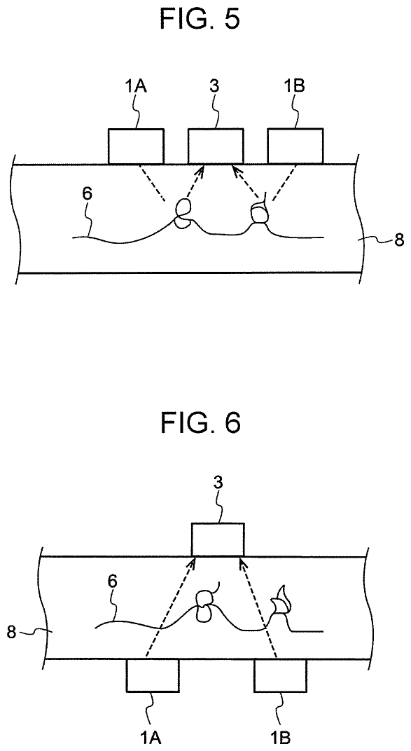

FIG. 5 illustrates an arrangement example of the light-emitting element 1A, the light-emitting element 1B, and the light-receiving element 3 in the photoelectric sensor 11. As illustrated in FIG. 5, the light-emitting element 1A, the light-emitting element 1B, and the light-receiving element 3 are arranged side by side in such a manner as to face one surface of the living body 8. In this case, the light-receiving element 3 receives the IR light and the red light that are reflected by the capillary 6 and the like of the living body 8.

However, the arrangement of the light-emitting element 1A, the light-emitting element 1B, and the light-receiving element 3 is not limited to the arrangement example illustrated in FIG. 5. For example, as illustrated in FIG. 6, the light-emitting element 1A, the light-emitting element 1B, and the light-receiving element 3 may be arranged in such a manner that the light-emitting elements 1A and 1B face the light-receiving element 3 with the living body 8 interposed between the light-emitting elements 1A and 1B and the light-receiving element 3. In this case, the light-receiving element 3 receives the IR light and the red light that passes through the living body 8.

Here, as an example, the light-emitting element 1A and the light-emitting element 1B will be described as surface emitting laser elements such as, for example, vertical cavity surface emitting lasers (VCSEL). However, each of the light-emitting elements 1A and 1B is not limited to a surface emitting laser element and may be an edge emitting laser element. Alternatively, each of the light-emitting elements 1A and 1B may be a light emitting diode (LED).

The photoelectric sensor 11 includes a clip (not illustrated) that is used for attaching the photoelectric sensor 11 to a body part of a subject, and the photoelectric sensor 11 is attached to the subject by using the clip (not illustrated) in such a manner that the photoelectric sensor 11 is in contact with the body surface of the subject, so that the IR light and the red light do not leak from the photoelectric sensor 11 to the outside. In order to cause the light-receiving element 3 to receive, as accurately as possible, the IR light and the red light that are reflected by the living body 8 of the subject or that passes through the living body 8 of the subject, the photoelectric sensor 11 may be positioned so as to be in contact with the body surface of the subject. However, the photoelectric sensor 11 may be disposed at a position that is spaced apart from the body surface and that is within an area in which the IR light and the red light, which are reflected by the living body 8 of the subject or which passes through the living body 8 of the subject, are received by the light-receiving element 3.

The photoelectric sensor 11 converts the amount of the IR light received by the light-receiving element 3 and the amount of the red light received by the light-receiving element 3 into, for example, voltage values and informs the pulse-wave processing unit 12 of the voltage values.

The light-emitting element 1A and the light-emitting element 1B each emit a predetermined amount of light, and thus, the amount of the IR light absorbed by the living body 8 and the amount of the red light absorbed by the living body 8 are determined from the amount of the IR light received by the photoelectric sensor 11 and the amount of the red light received by the photoelectric sensor 11.

Thus, the pulse-wave processing unit 12 generates, by using the received amounts of the IR light and the red light received from the photoelectric sensor 11, a pulse-wave signal indicating the pulse wave of the subject that is obtained from the IR light and a pulse-wave signal indicating the pulse wave of the subject that is obtained from the red light. The pulse-wave processing unit 12 amplifies voltage values that correspond to the received amounts of the IR light and the red light in such a manner that the voltage values are within a predetermined range that is suitable for generation of the pulse-wave signals. Then, the pulse-wave processing unit 12 generates the pulse-wave signals and removes a noise component from the pulse-wave signals by using a commonly known filter or the like.

The pulse-wave processing unit 12 informs the oxygen-saturation measuring unit 14 of the generated pulse-wave signals.

Upon reception of the pulse-wave signals from the pulse-wave processing unit 12, the oxygen-saturation measuring unit 14 measures the oxygen saturation of the subject by using the received pulse-wave signals. More specifically, the oxygen-saturation measuring unit 14 calculates, by using the pulse-wave signals, the change ΔA_{IR} of the amount of the absorbed IR light and the change ΔA_{Red} of the amount of the absorbed red light due to changes in the thickness of the artery 4 in accordance with Formula (1). Then, the oxygen-saturation measuring unit 14 measures the oxygen saturation of the subject from, for example, Formula (2) by using the calculated change ΔA_{IR} and the calculated change ΔA_{Red} and informs the oxygen-circulation-time measuring unit 17 of the measured oxygen saturation.

As an example, a case where the oxygen-saturation measuring unit 14 measures the oxygen saturation of a subject will be described below. The oxygen-saturation measuring unit 14 may measure any value as long as the value indicates temporal changes in the oxygen saturation of the subject. For example, the oxygen-saturation measuring unit 14 may measure a value such as a reciprocal of the oxygen saturation or the ratio between the change ΔA_{Red} and the change ΔA_{IR} that correlates with temporal changes in the oxygen saturation.

The receiving unit 13 is an example of a reception unit that receives the breathing state of a subject. More specifically, when the receiving unit 13 receives an instruction to inform stoppage of breathing via an input device that is operated by a subject or a measurer such as a healthcare professional who measures biological information of the subject, the receiving unit 13 assumes that the subject has stopped breathing. When the receiving unit 13 receives an instruction to inform resumption of breathing from the input device, the receiving unit 13 assumes that the subject has resumed breathing.

Then, the receiving unit 13 informs the notifying unit 16 of the breathing state of the subject, such as stoppage of breathing or resumption of breathing.

The timer 15 is an example of a measuring device that measures time and measures an accumulated time from a specified point in time.

The notifying unit 16 notifies the subject of the timing at which exhalation is to be started, the timing at which inhalation is to be started, the timing at which breathing is to be stopped, and the timing at which breathing is to be resumed in such a manner that the accuracy with which the oxygen saturation is measured is equal to or higher than a predetermined accuracy.

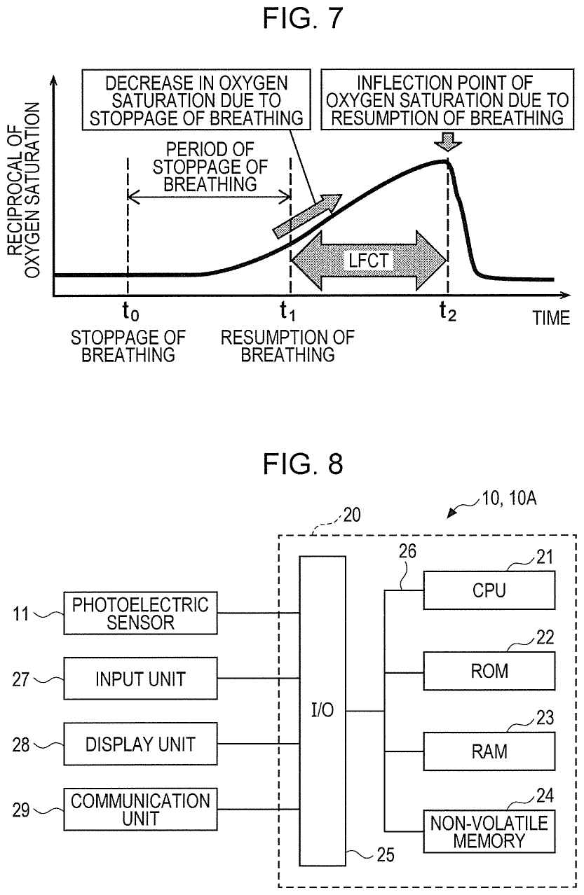

The graph in FIG. 7 illustrates an example of changes in the oxygen saturation in blood in a specific body part of a subject. In the graph, the horizontal axis denotes time, and the vertical axis denotes reciprocal of oxygen saturation.

When the subject stops breathing at time t₀, the oxygen saturation in blood in the subject starts decreasing. Even if the subject resumes breathing after a specified period of time has passed, the specified period of time being predetermined as the period of time during which the subject stops breathing, (at time t₁), it takes time for oxygen that is taken into the blood as a result of resumption of breathing to reach the specific body part from the lungs, and thus, the oxygen saturation in the blood in the subject keeps decreasing after time t₁. The oxygen taken in the blood as a result of resumption of breathing eventually reaches the specific body part from the lungs, and thus, the oxygen saturation in the blood in the subject starts increasing. The point in time at which the oxygen saturation in the blood turns from a decrease to an increase will be referred to as an “inflection point”. When the time at which the inflection point appears is referred to as time t₂, an oxygen circulation time is denoted by the difference between time t₁ and time t₂.

In other words, the oxygen circulation time corresponds to the time taken for oxygen to be transported from the lungs to a specific body part and is also called “oxygen transportation time”.

The accuracy with which the oxygen circulation time is measured from the oxygen saturation is likely to vary due to variations in the breathing state before breathing is stopped and variations in the period during which breathing is stopped, and thus, a breathing rhythm (hereinafter referred to as “reference breathing rhythm”) that functions as a reference suitable for measurement of the oxygen circulation time is predetermined.

Here, “breathing rhythm” is the breathing state expressed by using the period of time and the amount of exhalation and the period of time and the amount of inhalation and is represented by a waveform along the time axis. The reference breathing rhythm defines components that are used when breathing is represented by a waveform, the components including the timing at which exhalation is started, the intensity of exhalation, the length of exhalation, the timing at which inhalation is started, the intensity of inhalation, the length of inhalation, the timing at which breathing is stopped, the length of time during which breathing is stopped, the timing at which breathing is resumed, the number of times breathing is performed before breathing is stopped, and the number of times breathing is performed after breathing is resumed. The reference breathing rhythm is an example of a reference rhythm according to the present exemplary embodiment. Note that, in the reference breathing rhythm, a period of time that is specified as the period of time during which breathing is to be stopped is particularly called “specified period”.

The reference breathing rhythm is determined beforehand by, for example, an experiment using the actual apparatus that corresponds to the biological-information measuring apparatus 10 or a computer simulation based on the design specification of the biological-information measuring apparatus 10 in such a manner that the accuracy with which the oxygen circulation time is measured by the biological-information measuring apparatus 10 is equal to or higher than a predetermined accuracy.

The notifying unit 16 notifies, in accordance with the reference breathing rhythm, the subject of a rhythm that guides the subject in such a manner that the breathing rhythm of the subject becomes close to a rhythm with which the subject's nervousness associated with the measurement is relieved until the notifying unit 16 instructs the subject to stop breathing. After the notifying unit 16 has instructed the subject to stop breathing, the notifying unit 16 instructs the subject to resume breathing in such a manner that the period of time during which the subject stops breathing becomes close to the specified period. In addition, when the notifying unit 16 receives an instruction to notify resumption of breathing from the input device, the notifying unit 16 also notifies the oxygen-circulation-time measuring unit 17 that the subject has resumed breathing. Note that the notifying unit 16, which notifies the subject of the reference breathing rhythm, is an example of an informing unit according to the present exemplary embodiment.

Upon receiving information indicating that the subject has resumed breathing from the notifying unit 16, the oxygen-circulation-time measuring unit 17 stores the time at which the oxygen-circulation-time measuring unit 17 receives the information regarding resumption of breathing as time t₁. Then, the oxygen-circulation-time measuring unit 17 monitors the oxygen saturation that is measured by the oxygen-saturation measuring unit 14 and detects the inflection point of the oxygen saturation. The oxygen-circulation-time measuring unit 17 stores the time at which the inflection point of the oxygen saturation is detected as time t₂ and measures the period of time that is determined by the difference between time t₁ and time t₂ as the oxygen circulation time. Note that the wording “detects the inflection point” includes the case of detecting a position slightly offset from the inflection point within a range that does not substantially affect the measurement of the oxygen circulation time.

Subsequently, the oxygen-circulation-time measuring unit 17 informs the cardiac-output measuring unit 18 of the measured oxygen circulation time. As described above, the oxygen-circulation-time measuring unit 17 is an example of a measuring unit that measures oxygen circulation time.

Note that a body part at which the oxygen circulation time is measured is determined by a position on the subject to which the photoelectric sensor 11 is attached, and in the present exemplary embodiment, as an example, the photoelectric sensor 11 is attached to a distal body part of the subject. More specifically, the photoelectric sensor 11 is attached to a tip of a digit, and the oxygen circulation time in the case where oxygen is transported from the lungs to the tip of the digit is measured. This is because the tip of the digit is farther from the lungs than the other body parts are, and accordingly, the oxygen circulation time is longer, so that the oxygen circulation time may be obtained with an accuracy higher than that in the case where the photoelectric sensor 11 is attached to one of the other body parts. Note that the term “distal body part” refers to a body part that is located further toward the distal side than the neck, the shoulders, and the hip joint are in the body of the subject.

Thus, the oxygen circulation time between the lungs and a tip of a digit may sometimes be particularly referred to as “lung-to-finger circulation time (LFCT)”. Also in the present exemplary embodiment, although a case where the photoelectric sensor 11 is attached to a tip of a digit of a subject and where the LFCT is measured by the oxygen-circulation-time measuring unit 17 will be described, a body part to which the photoelectric sensor 11 is attached is not limited to a tip of a digit. The photoelectric sensor 11 may be attached to any body part of a subject as long as errors in the measurement of the oxygen circulation time are within a predetermined range. Note that, although the term “tip of a digit” refers to a fingertip or a thumb tip of a subject, the photoelectric sensor 11 may be attached to a tip of a toe of the subject.

The cardiac-output measuring unit 18 measures the cardiac output of a subject by using the LFCT received from the oxygen-circulation-time measuring unit 17. The cardiac output is calculated by using, for example, an arithmetic expression that is obtained beforehand and that represents the relationship between the LFCT and the cardiac output.

Note that the cardiac-output measuring unit 18 may measure information related to the cardiac output other than the cardiac output. The wording “information related to the cardiac output” refers to information that is considered to be correlated with the cardiac output and includes, for example, a cardiac index and a stroke volume.

The “cardiac index” is a value that is obtained by dividing the cardiac output of a subject by the body surface area of the subject so as to accommodate variations in cardiac output between subjects due to physical differences between the subjects. The “stroke volume” is a value that indicates the amount of blood that is pumped into the artery 4 by the heart in a single contraction and is obtained by dividing the cardiac output by the number of beats of the heart per minute.

The above-described biological-information measuring apparatus 10 includes, for example, a computer. FIG. 8 is a diagram illustrating a configuration example of a principal portion in an electrical system of the biological-information measuring apparatus 10 that includes a computer 20.

The computer 20 includes a central processing unit (CPU) 21, read only memory (ROM) 22, random access memory (RAM) 23, a non-volatile memory 24, and an input/output (I/O) interface 25 that function as the informing unit and the measuring unit according to the present exemplary embodiment. The CPU 21, the ROM 22, the RAM 23, the non-volatile memory 24, and the I/O interface 25 are connected to one another via a bus 26. Note that there is no limitation on an operation system that is used by the computer 20.

The non-volatile memory 24 is an example of a memory device that maintains information stored therein even when supply of power to the non-volatile memory 24 is discontinued, and for example, a semiconductor memory is used. However, the non-volatile memory 24 may be a hard disk.

For example, the photoelectric sensor 11, an input unit 27, a display unit 28, and a communication unit 29 are connected to the I/O interface 25.

The photoelectric sensor 11 and the I/O interface 25 are connected to each other by a wired or wireless connection. Note that the biological-information measuring apparatus 10 and the photoelectric sensor 11 may be provided so as to be separated from each other, or the biological-information measuring apparatus 10 and the photoelectric sensor 11 may be accommodated in the same housing so as to be integrated with each other.

The input unit 27 is, for example, a unit that receives an instruction from a subject and informs the CPU 21 of the instruction. The input unit 27 includes, for example, a button, a touch panel, a keyboard, and a mouse. As an example, the input unit 27 includes a button for indicating stoppage of breathing and a button for indicating resumption of breathing.

Accordingly, a subject presses the buttons so as to inform the biological-information measuring apparatus 10 of stoppage of breathing and resumption of breathing. In the following description, the button for indicating stoppage of breathing will be referred to as “breathing stoppage button”, and the button for indicating resumption of breathing will be referred to as “breathing resumption button”.

Note that the subject does not necessarily indicate stoppage of breathing and resumption of breathing by using such buttons and may inform the CPU 21 of stoppage of breathing and resumption of breathing by, for example, touching the touch panel, pressing the keyboard or operating the mouse. In addition, the timing at which the subject stops breathing and the timing at which the subject resumes breathing are not necessarily indicated by the subject or a different person, and the biological-information measuring apparatus 10 may indicate these timings by using a counter, a timer, or the like as will be described later.

The display unit 28 is a unit that visually displays, for example, information processed by the CPU 21. As the display unit 28, for example, a display device such as a liquid crystal display, an electro luminescence (EL) display, or a projector is used.

Note that the biological-information measuring apparatus 10 does not necessarily include the display unit 28, and for example, any type of unit may be connected to the I/O interface 25 as long as the unit informs a subject of the reference breathing rhythm.

For example, in the case of informing a subject of the reference breathing rhythm by using sound, a speaker unit may be connected to the I/O interface 25 instead of the display unit 28. In the case of informing a subject of the reference breathing rhythm by causing the subject to physically experience the rhythm, a vibration unit may be connected to the I/O interface 25 instead of the display unit 28. Alternatively, two or more units such as the display unit 28, a speaker unit, and a vibration unit that inform a subject of the reference breathing rhythm may be combined to inform a subject of the reference breathing rhythm.

The communication unit 29 includes a communication protocol that is used for connecting, for example, a communication line such as the Internet and the biological-information measuring apparatus 10 to each other and performs data communication between the biological-information measuring apparatus 10 and other external apparatuses that are connected to the communication line. The communication unit 29 may be compatible with, for example, the Bluetooth (Registered Trademark) that is used for communication in a line-of-sight distance of around about 10 m and the near field communication (NFC) that is used for communication in a short distance of around about 10 cm in addition to a wireless local area network (LAN).

The connection form to the communication line in the communication unit 29 may be a wired or wireless connection. If the biological-information measuring apparatus 10 does not need to communicate with other external apparatuses connected to the communication line, it is not necessary to connect the communication unit 29 to the I/O interface 25.

A unit to be connected to the I/O interface 25 is not limited to those described above, and for example, a unit that is different from those described above such as a printing unit that prints measurement results of biological information such as cardiac output and LFCT may be connected to the I/O interface 25.

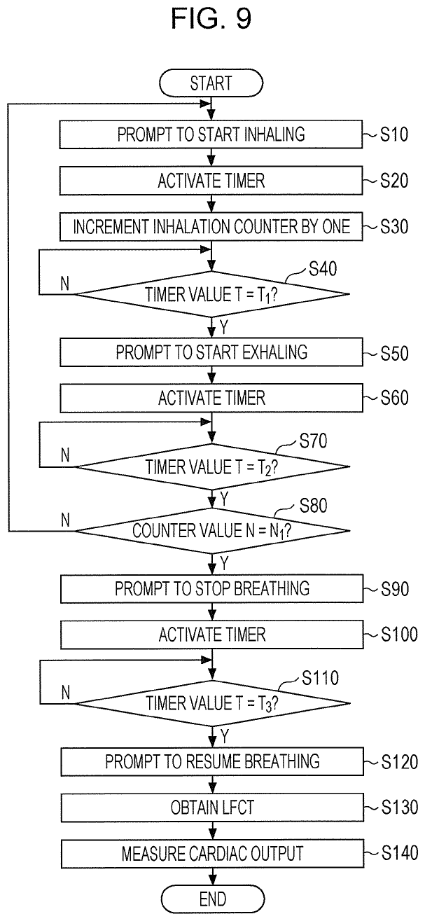

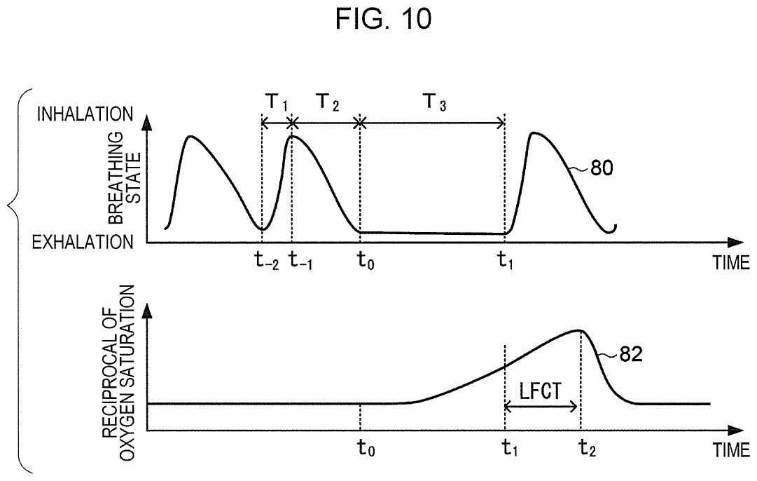

Operation of the biological-information measuring apparatus 10 will now be described with reference to FIG. 9 and FIG. 10.

FIG. 9 is a flowchart illustrating an example of the flow of measurement processing that is performed by the CPU 21 when the CPU 21 receives an instruction to measure cardiac output from a subject via the input unit 27 in a state where the photoelectric sensor 11 is attached to a tip of a digit of the subject.

FIG. 10 includes a graph illustrating the breathing state of a subject when the measurement processing illustrated in FIG. 9 is performed (a waveform 80) and a graph illustrating an example of changes in oxygen saturation (a waveform 82).

Upon receiving an instruction to measure cardiac output, the biological-information measuring apparatus 10 starts measuring the oxygen saturation of the subject and keeps measuring the oxygen saturation of the subject at least until measurement of the cardiac output is completed.

As mentioned above, although there is no limitation on the informing unit that informs the reference breathing rhythm, in this case, as an example, the display unit 28 is used to inform the subject of the reference breathing rhythm unless otherwise stated.

For example, a biological-information measurement program that defines the measurement processing is stored beforehand in the ROM 22 of the biological-information measuring apparatus 10. The CPU 21 of the biological-information measuring apparatus 10 reads the biological-information measurement program stored in the ROM 22 and performs the measurement processing.

First, in step S10, the CPU 21 informs the subject of the timing at which inhalation is to be started by controlling the display unit 28 such that the display unit 28 displays an image instructing the subject to start inhaling. By performing this step, the subject starts inhaling. For example, time t₋₂ illustrated in FIG. 10 is the time when the subject is instructed to start inhaling.

In step S20, The CPU 21 activates the timer 15 and measures the elapsed time from when the subject is instructed to start inhaling. The CPU 21 may measure the elapsed time by using, for example, a timer function embedded in the CPU 21 or may measure the elapsed time by using an external timer unit that is connected to the I/O interface 25.

In step S30, as the subjects starts inhaling, the CPU 21 increments an inhalation counter, which counts the number of inhalations, by one. The inhalation counter is stored in the RAM 23, and a counter value N of the inhalation counter is initialized with “0” each time the measurement processing is started. When a combination of a single inhalation and a single exhalation is considered as a single breathing, the inhalation counter indicates the number of inhalations and also is a counter that indicates the number of times breathing is performed.

In step S40, the CPU 21 determines whether a timer value T of the timer 15 activated in step S20 is equal to a threshold T₁. The threshold T₁ is a value that defines the length of inhalation in the reference breathing rhythm until the subject is instructed to stop breathing. The threshold T₁ is a value that is set beforehand so as to guide the subject to breathe in such a manner as to relieve the subject's nervousness associated with the measurement of cardiac output. The threshold T₁ is determined by, for example, an experiment using the actual apparatus that corresponds to the biological-information measuring apparatus 10 and is stored in, for example, the non-volatile memory 24.

When the subject is in a resting state, the subject does not inhale in such a manner as to reach their limit at which the subject is no longer able to inhale, that is, the subject does not take a full inhalation, and thus, the threshold T₁ may be set to such a value that the subject does not take a full inhalation and that the breathing state of the subject becomes close to the breathing state when the subject is in the resting state.

When the timer value T of the timer 15 is less than the threshold T₁, this indicates that the length of inhalation performed by the subject does not reach the period of time defined by the threshold T₁. Thus, the determination process in step S40 is repeatedly performed, and the timer value T of the timer 15 is monitored.

In contrast, when the timer value T of the timer 15 is equal to the threshold T₁, this indicates that the length of inhalation performed by the subject has reached the period of time defined by the threshold T₁, and thus, the processing continues to step S50.

In step S50, the CPU 21 informs the subject of the timing at which exhalation is to be started by controlling the display unit 28 such that the display unit 28 displays an image instructing the subject to start exhaling. By performing this step, the subject starts exhaling. For example, time t₋₁ illustrated in FIG. 10 is the time when the subject is instructed to start exhaling.

In step S60, the CPU 21 stops the timer 15, then activates the timer 15 again, and measures the elapsed time from when the subject is instructed to start exhaling.

In step S70, the CPU 21 determines whether the timer value T of the timer 15 activated in step S60 is equal to a threshold T₂. The threshold T₂ is a value that defines the length of exhalation in the reference breathing rhythm until the subject is instructed to stop breathing. The threshold T₂ is a value that is set beforehand so as to guide the subject to breathe in such a manner as to relieve the subject's nervousness associated with the measurement of cardiac output. The threshold T₂ is determined by, for example, an experiment using the actual apparatus that corresponds to the biological-information measuring apparatus 10 and is stored in, for example, the non-volatile memory 24.

When the subject is in the resting state, the subject does not exhale in such a manner as to reach their limit at which the subject is no longer capable of exhaling, that is, the subject does not take a full exhalation, and thus, the threshold T₂ may be set to such a value that the subject does not take a full exhalation and that the breathing state of the subject becomes close to the breathing state when the subject is in the resting state.

When the timer value T of the timer 15 is less than the threshold T₂, this indicates that the length of exhalation performed by the subject does not reach the period of time defined by the threshold T₂. Thus, the determination process in step S70 is repeatedly performed, and the timer value T of the timer 15 is monitored.

In contrast, when the timer value T of the timer 15 is equal to the threshold T₂, this indicates that the length of exhalation performed by the subject has reached the period of time defined by the threshold T₂, and thus, the processing continues to step S80.

Note that the threshold T₂ is set to be greater than the threshold T₁. As a result, the subject is led to breathe in such a manner that the length of exhalation is longer than the length of inhalation. In the case where the length of exhalation is longer than the length of inhalation, the parasympathetic nerve becomes more active than the sympathetic nerve, and thus, the nervousness is likely to be relieved.

In step S80, the CPU 21 determines whether the counter value N of the inhalation counter that is updated in step S30 is equal to a threshold N₁. The threshold N₁ is a value that defines the number of times breathing is performed during the period from the time when an instruction to measure cardiac output is received to the time when the subject is instructed to stop breathing, and the threshold N₁ is set to a value at which breathing is performed a predetermined number of times (e.g., about a few times to about a few dozen times) by which the subject's nervousness associated with the measurement of cardiac output is relieved. The threshold N₁ is stored in, for example, the non-volatile memory 24.

When the counter value N of the inhalation counter is less than the threshold N₁, the number of times the subject has performed breathing does not reach the number of times defined by the threshold N₁, and thus, the processing returns to step S10.

When the number of times the subject has performed breathing reaches the threshold N₁ by repeatedly performing step S10 to step S80, the processing continues to step S90.

In step S90, the CPU 21 informs the subject of the timing at which breathing is to be stopped by controlling the display unit 28 such that the display unit 28 displays an image instructing the subject to stop breathing. By performing this step, the subject stops breathing. In other words, time t₀ illustrated in FIG. 10 is the time when the subject is instructed to stop breathing. Note that the time when the image instructing the subject to stop breathing is displayed may be set as time t₀, or the time when the subject or a different person indicates, in response to the displayed image, that breathing has stopped by using the breathing stoppage button or the like may be set as time t₀.

In step S100, the CPU 21 stops the timer 15, then activates the timer 15 again, and measures the elapsed time from when the subject is instructed to stop breathing.

In step S110, the CPU 21 determines whether the timer value T of the timer 15 activated in step S100 is equal to a threshold T₃. The threshold T₃ is the specified period that specifies the period of time during which breathing is to be stopped. The threshold T₃ is determined by, for example, an experiment using the actual apparatus that corresponds to the biological-information measuring apparatus 10 or a computer simulation based on the design specification of the biological-information measuring apparatus 10 so as to improve the accuracy with which the oxygen circulation time is measured by the biological-information measuring apparatus 10 and is stored in, for example, the non-volatile memory 24.

When the timer value T of the timer 15 is less than the threshold T₃, the elapsed time from when the subject stops breathing does not reach the specified period, and thus, the determination process in step S110 is repeatedly performed, and the timer value T of the timer 15 is monitored.

In contrast, when the timer value T of the timer 15 is equal to the threshold T₃, the elapsed time from when the subject stops breathing has reached the specified period, and thus, the processing continues to step S120.

In step S120, the CPU 21 informs the subject of the timing at which breathing is to be resumed by controlling the display unit 28 such that the display unit 28 displays an image instructing the subject to resume breathing. By performing this step, the subject resumes breathing. In other words, time t₁ illustrated in FIG. 10 is the time when the subject is instructed to resume breathing. Note that the time when the image instructing the subject to resume breathing is displayed may be set as time t₁, or the time when the subject or a different person indicates, in response to the displayed image, breathing has resumed by using the breathing resumption button or the like may be set as time t₁.

In the case where the CPU 21 instructs the subject to stop breathing without considering the timing of the instruction, the subject may sometimes stop breathing immediately after inhaling. In this case, when the subject is instructed to resume breathing, the subject starts exhaling first. Thus, compared with the case where the subject starts inhaling first at the time of resumption of breathing, the LFCT that is measured becomes longer than the actual LFCT, and consequently, the accuracy of the LFCT measurement deteriorates.

However, since the CPU 21 instructs the subject to stop breathing after the subject has finished exhaling and before the subject starts inhaling, when the subject is instructed to resume breathing in step S120, the subject starts inhaling first.

The CPU 21 stores the time when the CPU 21 informs the subject of the timing at which breathing is to be resumed as time t₁ in the RAM 23.

In step S130, the CPU 21 monitors changes in the oxygen saturation. Then, the CPU 21 obtains time t₂ at which the inflection point of the oxygen saturation is detected and stores time t₂ in the RAM 23. The CPU 21 obtains the difference between time t₂, at which the inflection point of the oxygen saturation appears, and time t₁, at which breathing is resumed and which is stored in the RAM 23 in step S120, as the LFCT. More specifically, the CPU 21 may stop the timer 15 and then activate the timer 15 again after instructing the subject to resume breathing in step S120 and may obtain, as the LFCT, the elapsed time from when the CPU 21 instructs the subject to resume breathing until the inflection point of the oxygen saturation is detected.

In step S140, the CPU 21 measures the cardiac output from, for example, Formula (6) by using the LFCT obtained in step S130. In addition, the CPU 21 may calculate information related to cardiac output by using the measured cardiac output. In the manner described above, the measurement processing illustrated in FIG. 9 is completed.

Note that the breathing cycle when the subject is nervous is likely to be shorter than the breathing cycle when the subject is in the resting state. When the breathing cycle becomes short, the breathing rhythm changes, and thus, the accuracy of the LFCT measurement becomes lower than that when the subject is in the resting state. In addition, when the subject is in such a nervous state, the subject tends to breathe earlier than the timing at which the biological-information measuring apparatus 10 instructs the subject to breathe.

Thus, the biological-information measuring apparatus 10 may set the threshold t₁ and the threshold t₂ in such a manner that the breathing cycle of the subject is longer than the breathing cycle when the subject is in the resting state until the subject is instructed to stop breathing.

By guiding the breathing cycle of the subject to become longer than the breathing cycle when the subject is in the resting state, the breathing cycle shortened due to the nervousness may sometimes become close to the breathing cycle when the subject is in the resting state.

Note that the breathing cycles of subjects when the subjects are in the resting state may be obtained beforehand by attaching a breathing sensor to each of the subjects, and the obtained breathing cycles of the subjects may be stored in the non-volatile memory 24. The biological-information measuring apparatus 10 may autonomously set the threshold t₁ and the threshold t₂ in such a manner that, for example, a subject is informed of a breathing cycle that is longer than the breathing cycle when the subject is in the resting state. Alternatively, a user who uses the biological-information measuring apparatus 10 may set the threshold t₁ and the threshold t₂ in such a manner that a subject is informed of a breathing cycle that is longer than the breathing cycle when the subject is in the resting state.

The various parameters such as the threshold t₁, the threshold t₂, the threshold t₃, and the threshold N₁ may be set by using the input unit 27 or may be set by using an external apparatus via the communication unit 29.

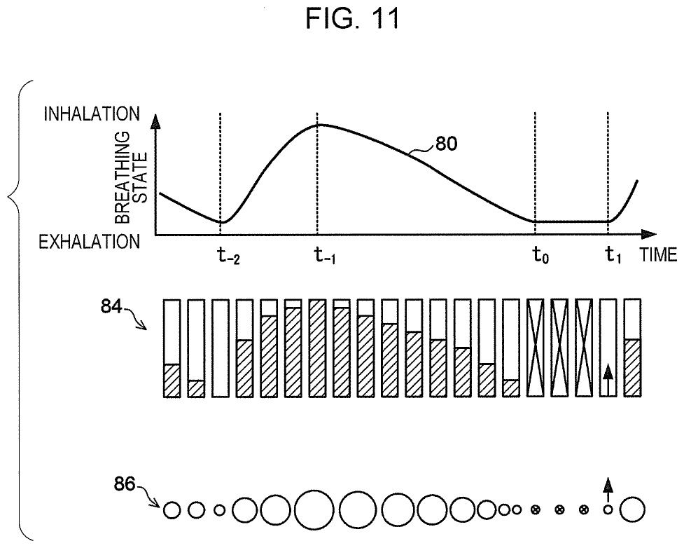

FIG. 11 is a diagram illustrating examples of the way of indicating the reference breathing rhythm that is displayed on the display unit 28 through the measurement processing illustrated in FIG. 9.

Regarding the way of indicating the reference breathing rhythm on the display unit 28, as illustrated in FIG. 11, the reference breathing rhythm is represented by, for example, the waveform 80, a bar graph 84, a circle graph 86, or the like.

In the examples illustrated in FIG. 11, the inflection point at which the waveform 80 turns from a decrease to an increase (also referred to as “local minimum point”: time t₋₂) indicates the timing at which inhalation is to be started, and the inflection point at which the waveform 80 turns from an increase to a decrease (also referred to as “local maximum point”: time t₋₁) indicates the timing at which exhalation is to be started. In addition, the first point in time (time t₀) at which the waveform 80 stops decreasing and remains unchanged indicates the timing at which breathing is to be stopped, and the point in time (time t₁) at which the waveform 80 turns from the state of remaining unchanged to an increase indicates the timing at which breathing is to be resumed.

In the case where the reference breathing rhythm is represented by the bar graph 84, the reference breathing rhythm is represented by using the height of a shaded portion of the bar graph 84. When the height of the shaded portion of the bar graph 84 turns from a decrease to an increase, this point in time (time t₋₂) is the timing at which inhalation is to be started, and when the height of the shaded portion of the bar graph 84 turns from an increase to a decrease, this point in time (time t₋₁) is the timing at which exhalation is to be started. In addition, when a cross mark is displayed after the height of the shaded portion of the bar graph 84 has decreased, this point in time (time t₀) is the timing at which breathing is to be stopped, and when the cross mark is changed to an upward arrow, this point in time (time t₁) is the timing at which breathing is to be resumed.

In the case where the reference breathing rhythm is represented by the circle graph 86, the reference breathing rhythm is represented by using the size of the circle graph 86. When the size of the circle graph 86 turns from a decrease to an increase, this point in time (time t₋₂) is the timing at which inhalation is to be started, and when the size of the circle graph 86 turns from an increase to a decrease, this point in time (time t₋₁) is the timing at which exhalation is to be started. In addition, when a cross mark is displayed after the size of the circle graph 86 has decreased, this point in time (time t₀) is the timing at which breathing is to be stopped, and when an upward arrow is displayed in the circle graph 86, this point in time (time t₁) the timing at which breathing is to be resumed.

FIG. 11 illustrates display examples of the reference breathing rhythm, and the way of indicating the reference breathing rhythm is not limited to the examples illustrated in FIG. 11, and any notification way may be employed as long as a subject is capable of visually recognizing the reference breathing rhythm. For example, textual information “inhale” may be displayed at the timing at which inhalation is to be started, and textual information “exhale” may be displayed at the timing at which exhalation is to be started. Alternatively, the remaining time until exhalation is started, the remaining time until inhalation is started, the remaining time until breathing is stopped, and the remaining time until breathing is resumed may be counted down and displayed by using numbers. In addition, a plurality of notification ways may be combined (e.g., a combination of the waveform 80 and the bar graph 84).

The biological-information measuring apparatus 10 may inform a subject of the reference breathing rhythm by using sound and vibration instead of using the display unit 28.

For example, a buzzer sound or a human voice is used as the sound used for informing a subject of the reference breathing rhythm, and the biological-information measuring apparatus 10 causes a speaker unit (not illustrated) to emit different sounds in order to inform a subject of the timing at which exhalation is to be started, the timing at which inhalation is to be started, the timing at which breathing is to be stopped, and the timing at which breathing is to be resumed. More specifically, in the case of using the sound of a buzzer, for example, at least one of the length of the buzzer sound, the pitch of the buzzer sound, and the number of times the buzzer beeps is changed. In the case of using a human voice, the contents of the voice guidance that is given to a subject are changed (e.g., “inhale” and “exhale”).

As described above, the biological-information measuring apparatus 10 according to the present exemplary embodiment informs a subject of a reference rhythm in order to guide the breathing rhythm of the subject to become close to a rhythm suitable for measurement of biological information, such as cardiac output, the measured value of which varies in accordance with the breathing state.

### Second Exemplary Embodiment

Although the biological-information measuring apparatus 10 according to the first exemplary embodiment informs a subject of the reference breathing rhythm that is suitable for measurement of biological information that is measured and the value of which varies in accordance with the breathing state of the subject, the biological-information measuring apparatus 10 does not include a unit that determines whether the breathing rhythm of the subject is close to the reference breathing rhythm.

In the second exemplary embodiment, a biological-information measuring apparatus 10A that detects the breathing rhythm of a subject and adjusts the reference breathing rhythm in accordance with the breathing rhythm of the subject will be described.

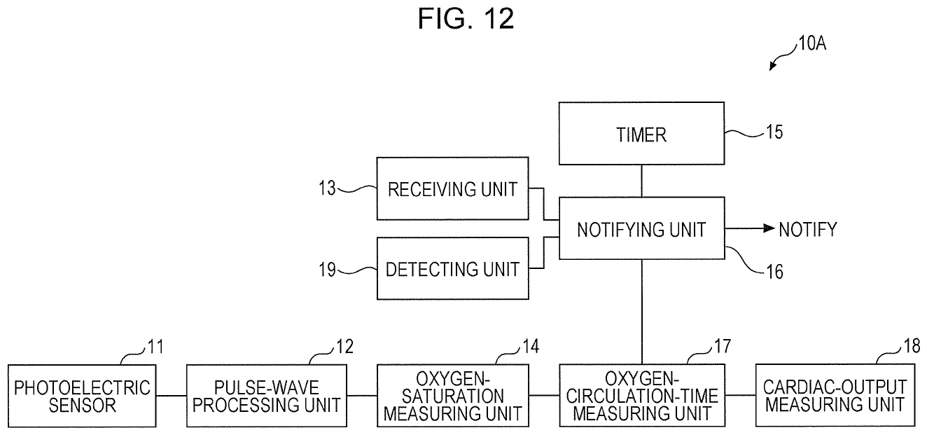

FIG. 12 is a diagram illustrating a configuration example of the biological-information measuring apparatus 10A. The difference between the configuration of the biological-information measuring apparatus 10A illustrated in FIG. 12 and the configuration of the biological-information measuring apparatus 10 illustrated in FIG. 4 is that the biological-information measuring apparatus 10A further includes a detecting unit 19, and the rest of the configuration of the biological-information measuring apparatus 10A is the same as that of the biological-information measuring apparatus 10.

The detecting unit 19 detects the breathing rhythm of a subject and informs the notifying unit 16 of the detected breathing rhythm. More specifically, the detecting unit 19 detects the breathing rhythm of the subject by obtaining a sensor value of a measurement sensor, such as a breathing sensor that is attached to the subject, that measures a breathing rhythm.

Upon receiving the breathing rhythm of the subject from the detecting unit 19, the notifying unit 16 adjusts the reference breathing rhythm, which is predefined by the various parameters such as the threshold t₁, the threshold t₂, the threshold t₃, and the threshold N₁, in accordance with the breathing rhythm of the subject and notifies the subject of the adjusted reference breathing rhythm.

Note that the configuration of a principal portion of an electrical system of the biological-information measuring apparatus 10A is the same as the configuration example of the principal portion in the electrical system of the biological-information measuring apparatus 10, which is illustrated in FIG. 8.

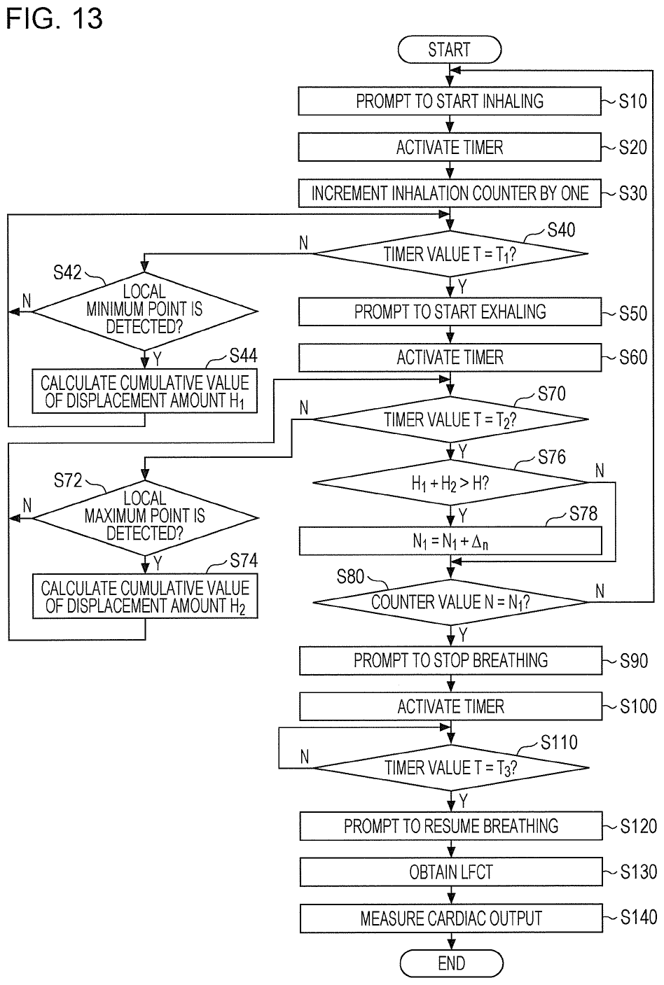

Operation of the biological-information measuring apparatus 10A will now be described with reference to FIG. 13.

FIG. 13 is a flowchart illustrating an example of the flow of measurement processing that is performed by the CPU 21 when the CPU 21 receives an instruction to measure cardiac output from a subject via the input unit 27 in a state where the photoelectric sensor 11 is attached to a tip of a digit of the subject.

The difference between the flowchart illustrated in FIG. 13 and the flowchart illustrated in FIG. 9 of the measurement processing that is performed by the biological-information measuring apparatus 10 is that the flowchart illustrated in FIG. 13 further includes steps S42, S44, S72, S74, S76, and S78, and the rest of the processes of the flowchart illustrated in FIG. 13 are the same as those of the flowchart illustrated in FIG. 9.

The biological-information measuring apparatus 10A detects the breathing rhythm of a subject before receiving an instruction to measure cardiac output. Upon receiving the instruction to measure cardiac output, the biological-information measuring apparatus 10A starts measuring the oxygen saturation of the subject and keeps measuring the oxygen saturation of the subject at least until the measurement of the cardiac output is completed.

When it is determined, in the determination process in step S40, that the length of inhalation performed by the subject does not reach the period of time defined by the threshold T t₁, step S42 is performed.

In step S42, the CPU 21 refers to the waveform 80 of breathing performed by the subject that is obtained from the detected breathing rhythm of the subject and determines whether there is a local minimum point corresponding to the point in time at which the subject starts inhaling within a first monitoring period that is set beforehand so as to across the time when the subject is informed of the timing at which inhalation is to be started in step S10.

In the case where the local minimum point is not present in the first monitoring period, the subject, who has been exhaling, has not yet started inhaling, and thus, the processing returns to step S40 so as to keep monitoring the breathing state of the subject.

In contrast, in the case where the local minimum point is present in the first monitoring period, this indicates that the subjects has started inhaling, and thus, the processing continues to step S44.

In step S44, the CPU 21 calculates a displacement amount H₁ between the time when the local minimum point appears and the time when the subject is informed of the timing at which inhalation is to be started in step S10 and stores the displacement amount H₁ in the RAM 23. Note that, since the first monitoring period is set so as to across the time when the subject is informed of the timing at which inhalation is to be started in step S10, even if the subject starts inhaling before the subject is instructed to start inhaling, the displacement amount H₁ is obtained. Thus, in the case where the center of the first monitoring period is set to the time when the subject is informed of the timing at which inhalation is to be started, the first monitoring period may be set to a period of time that is twice or more of the conceivable maximum value of the displacement amount H₁.

When it is determined, in the determination process in step S70, that, the length of exhalation performed by the subject does not reach the period of time defined by the threshold t₂, step S72 is performed.

In step S72, the CPU 21 refers to the waveform 80 of breathing performed by the subject that is obtained from the detected breathing rhythm of the subject and determines whether there is a local maximum point corresponding to the point in time at which the subject starts exhaling within a second monitoring period that is set beforehand so as to across the time when the subject is informed of the timing at which exhalation is to be started in step S50.

In the case where the local maximum point is not present in the second monitoring period, the subject, who has been inhaling, has not yet started exhaling, and thus, the processing returns to step S70 so as to keep monitoring the breathing state of the subject.

In contrast, in the case where the local maximum point is present in the second monitoring period, this indicates that the subjects has started exhaling, and thus, the processing continues to step S74.

In step S74, the CPU 21 calculates a displacement amount H₂ between the time when the local maximum point appears and the time when the subject is informed of the timing at which exhalation is to be started in step S50 and stores the displacement amount H₂ in the RAM 23. Note that, since the second monitoring period is set so as to across the time when the subject is informed of the timing at which exhalation is to be started in step S50, even if the subject starts exhaling before the subject is instructed to start exhaling, the displacement amount H₂ is obtained. Thus, in the case where the center of the second monitoring period is set to the time when the subject is informed of the timing at which exhalation is to be started, the second monitoring period may be set to a period of time that is twice or more of the conceivable maximum value of the displacement amount H₂.

When it is determined, in the determination process in step S70, that, the length of exhalation performed by the subject has reached the period of time defined by the threshold t₂, step S76 is performed.

In step S76, the CPU 21 determines whether the sum (hereinafter referred to as “breathing displacement amount”) of the displacement amount H₁, which is calculated in step S44, and the displacement amount H₂, which is calculated in step S74, is greater than a predetermined displacement amount H. In other words, the CPU 21 determines whether the breathing rhythm of the subject is close to the reference rhythm from the breathing displacement amount. The predetermined displacement amount H is set to such a value that, when the breathing displacement amount is equal to or less than the predetermined displacement amount H, measurement of LFCT is performed by the biological-information measuring apparatus 10A with an accuracy equal to or higher than a predetermined accuracy.

When the breathing displacement amount is greater than the predetermined displacement amount H, that is, when the breathing rhythm of the subject deviates from the reference rhythm, the processing continues to step S78.

When the breathing rhythm of the subject deviates from the reference rhythm, the subject's nervousness associated with the measurement of cardiac output may sometimes not be relieved even if the subject repeats breathing the number of times defined by the threshold N₁, whereas in the case where the breathing rhythm of the subject is close to the reference rhythm, the subject's nervousness is relieved.

Thus, in step S78, the CPU 21 sets the value of the threshold N₁ to be greater than the most recent value. In this case, the CPU 21 may increase the value to be added to the threshold value N₁ as the difference between the predetermined displacement amount H and the breathing displacement amount increases. In other words, the number of times breathing is performed by the subject is adjusted in such a manner that the length of time from when notification of the reference breathing rhythm is started until the subject is instructed to stop breathing increases as the amount of deviation between the breathing rhythm of the subject and the reference rhythm increases.

In contrast, when the breathing displacement amount is equal to or less than the predetermined displacement amount H, that is, when the breathing rhythm of the subject is close to the reference rhythm, the processing continues to step S80 without performing the process of step S78.

In the subsequent steps, as described above, when the number of times breathing is performed by the subject reaches the threshold N₁, the subject is instructed to stop breathing and then instructed to resume breathing, and the LFCT is obtained. After that, the cardiac output is measured, and the measurement processing illustrated in FIG. 13 is completed.

In the present exemplary embodiment, although the breathing displacement amount is the sum of the displacement amount H₁ and the displacement amount H₂ as an example, a different value that quantitatively indicates the amount of deviation between the breathing rhythm of the subject and the reference rhythm may be used. For example, one of the displacement amounts H₁ and H₂ may be set as the breathing displacement amount, and alternatively, the cumulative value of the displacement amount H₁ or the cumulative value of the displacement amount H₂ may be set as the breathing displacement amount.

In addition, when the difference between the predetermined displacement amount H and the breathing displacement amount decreases after the breathing displacement amount has exceeded the predetermined displacement amount H, the value of the threshold N₁ may be adjusted so as to be less than the most recent value.

When the difference between the predetermined displacement amount H and the breathing displacement amount exceeds a predetermined acceptable range, the CPU 21 may issue an instruction prompting the subject to breathe to the reference rhythm.

As described above, the biological-information measuring apparatus 10A according to the present exemplary embodiment detects the breathing rhythm of a subject and informs the subject of the reference breathing rhythm that has been adjusted in accordance with the detected breathing rhythm of the subject.

Although the present disclosure has been described above by using the exemplary embodiments, the present disclosure is not limited to the exemplary embodiments. Various changes or improvements may be made to the exemplary embodiments within the gist of the present disclosure, and exemplary embodiments obtained by making such changes or improvements to the above exemplary embodiments are also included in the technical scope of the present disclosure. For example, the order of the processes may be changed within the gist of the present disclosure.

In addition, in each of the exemplary embodiments, although a case where the measurement processing is implemented via software has been described as an example, processing equivalent to that illustrated by the flowchart in FIG. 9 and that illustrated by the flowchart in FIG. 13 may be implemented in, for example, an application specific integrated circuit (ASIC) and may be performed by hardware. In this case, speeding up of the detection process may be achieved.

In the above-described exemplary embodiments, although a case where the biological-information measurement program is installed in the ROM 22 has been described, the present disclosure is not limited to this case. The biological-information measurement program according to the present disclosure may also be provided by being recorded in a computer-readable storage medium. For example, the biological-information measurement program according to the exemplary embodiments may be provided by being recorded in an optical disc such as a compact disc (CD)-ROM or a digital versatile disc (DVD)-ROM. Alternatively, the biological-information measurement program according to the exemplary embodiments may be provided by being recorded in a semiconductor memory such as a universal serial bus (USB) memory or a flash memory. Each of the biological-information measuring apparatuses 10 and 10A may obtain the biological-information measurement program according to the exemplary embodiments from an external apparatus connected to the communication line via the communication unit 29.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A biological-information measuring apparatus comprising:
an informing unit that informs a subject whose biological information is to be measured of a reference rhythm that is predetermined as a rhythm suitable for measurement of the biological information, a measured value of which varies in accordance with a breathing state of the subject, in such a manner that a breathing rhythm of the subject becomes close to the reference rhythm; and
a measuring unit that measures the biological information of the subject who performs breathing to the reference rhythm, which is given to the subject by the informing unit.

2. The biological-information measuring apparatus according to claim 1,
wherein the measuring unit measures biological information related to a cardiac function of the subject.

3. The biological-information measuring apparatus according to claim 2,
wherein the measuring unit measures an oxygen circulation time from a value indicating a concentration of oxygen in blood of the subject.

4. The biological-information measuring apparatus according to claim 1,
wherein the informing unit informs the subject of the reference rhythm in such a manner that the subject stops breathing after finishing exhaling and before starting inhaling.

5. The biological-information measuring apparatus according to claim 4,
wherein the informing unit informs the subject of the reference rhythm in such a manner that a way of informing the subject of a timing at which breathing is to be stopped in the reference rhythm is different from a way of informing the subject of a timing at which exhalation is to be started in the reference rhythm and a way of informing the subject of a timing at which inhalation is to be started in the reference rhythm.

6. The biological-information measuring apparatus according to claim 5,
wherein the informing unit informs the subject of the reference rhythm in a cycle that is longer than a breathing cycle of the subject when the subject is in a resting state, the breathing cycle being obtained beforehand, until the informing unit informs the subject of the timing at which breathing is to be stopped.

7. The biological-information measuring apparatus according to claim 5,
wherein the informing unit informs the subject of the reference rhythm in such a manner that a length of exhalation is longer than a length of inhalation until the informing unit informs the subject of the timing at which breathing is to be stopped.

8. The biological-information measuring apparatus according to claim 4, further comprising:
a detecting unit that detects the breathing rhythm of the subject,
wherein the informing unit informs the subject of the reference rhythm in which a length of time from when the informing unit starts informing the subject of the reference rhythm until the informing unit informs the subject of the timing at which breathing is to be stopped is adjusted in accordance with an amount of deviation between the breathing rhythm of the subject detected by the detecting unit and the reference rhythm.

9. The biological-information measuring apparatus according to claim 8,
wherein the informing unit informs the subject of the reference rhythm in such a manner that the length of time from when the informing unit starts informing the subject of the reference rhythm until the informing unit informs the subject of the timing at which breathing is to be stopped increases as the amount of deviation between the breathing rhythm of the subject detected by the detecting unit and the reference rhythm increases.

10. The biological-information measuring apparatus according to claim 8,
wherein the informing unit instructs the subject to breathe to the reference rhythm when the amount of deviation exceeds an acceptable range.

11. A non-transitory computer readable medium storing a program causing a computer to function as each of the units included in the biological-information measuring apparatus according to claim 1.
